(19) United States
(12) Patent Application Publication
(10) Pub. No.: US 2023/0108049 A1
(43) Pub. Date: Apr. 6, 2023

(54) ORAL FAST-DISPERSING DOSAGE FORM OF RIMEGEPANT

(71) Applicant: BIOHAVEN PHARMACEUTICAL HOLDING COMPANY LTD., New Haven, CT (US)

(72) Inventors: Vladimir CORIC, New Haven, CT (US); Marianne FROST, North Haven, CT (US); Robert CROOP, Newton Square, PA (US); Charles M. CONWAY, Cheshire, CT (US)

(21) Appl. No.: 17/801,295

(22) PCT Filed: Feb. 26, 2021
(86) PCT No.: PCT/US21/19825

§ 371 Date: Aug. 22, 2022

Related U.S. Application Data
(60) Provisional application No. 62/982,456, filed on Feb. 27, 2020.

(51) Int. Cl. A61K 31/4545; A61K 9/00; A61P 25/06; A61K 9/20

(52) U.S. Cl. CPC A61K 31/4545; A61K 9/0053; A61P 25/06; A61K 9/20

(57) ABSTRACT

Provided is a fast-dispersing dosage form for oral delivery, wherein the fast-dispersing dosage form includes a rimegepant. Also provided is a method for delivering rimegepant to a subject, wherein the method includes orally administering to the subject a fast-dispersing dosage form including rimegepant.

### CROSS-REFERENCE TO RELATED APPLICATIONS

This application claims priority to U.S. Provisional Application No. 62/982,456 filed Feb. 27, 2020 and all the benefits accruing therefrom under 35 U.S.C. § 119, the content of which is incorporated herein in its entirety by reference.

### FIELD OF THE INVENTION

The present invention relates to pharmaceutical compositions of (5S,6S,9R)-5-amino-6-(2,3-difluorophenyl)-6,7,8,9-tetrahydro-5H-cyclohepta[b]pyridin-9-yl 4-(2-oxo-2,3-dihydro-1H-imidazo[4,5-b]pyridin-1-yl)-1-piperidinecarboxylate (rimegepant), a calcitonin gene-related peptide (CGRP) receptor antagonist. Specifically, the present invention relates to an oral fast-dispersing dosage form of rimegepant which may be used for treating CGRP-related disorders such as migraine.

### BACKGROUND OF THE INVENTION

Migraine is a chronic and debilitating disorder characterized by recurrent attacks lasting four to 72 hours with multiple symptoms, including typically one-sided, pulsating headaches of moderate to severe pain intensity that are associated with nausea or vomiting, and/or sensitivity to sound (phonophobia) and sensitivity to light (photophobia). Migraines are often preceded by transient neurological warning symptoms, known as auras, which typically involve visual disturbances such as flashing lights, but may also involve numbness or tingling in parts of the body. Migraine is both widespread and disabling. The Migraine Research Foundation ranks migraine as the world's third most prevalent illness, and the Global Burden of Disease Study 2015 rates migraine as the seventh highest specific cause of disability worldwide. According to the Migraine Research Foundation, in the United States, approximately 36 million individuals suffer from migraine attacks. While most sufferers experience migraine attacks once or twice per month, more than 4 million people have chronic migraine, defined as experiencing at least 15 headache days per month, of which at least eight are migraine, for more than three months. Others have episodic migraine, which is characterized by experiencing less than 15 migraine days per month. People with episodic migraine may progress to chronic migraine over time. Migraine attacks can last four hours or up to three days. More than 90% of individuals suffering from migraine attacks are unable to work or function normally during a migraine attack, with many experiencing comorbid conditions such as depression, anxiety and insomnia. Also, those suffering from migraine often have accompanying nausea and have an aversion to consuming food or liquids during an attack.

CGRP (calcitonin gene-related peptide) is a 37 amino acid neuropeptide, which belongs to a family of peptides that includes calcitonin, adrenomedullin and amylin. In humans, two forms of CGRP (α-CGRP and β-CGRP) exist and have similar activities. They vary by three amino acids and exhibit differential distribution. At least two CGRP receptor subtypes may also account for differential activities. The CGRP receptor is located within pain-signaling pathways, intracranial arteries and mast cells and its activation is thought to play a causal role in migraine pathophysiology. For example, research and clinical studies have shown: serum levels of CGRP are elevated during migraine attacks, infusion of intravenous CGRP produces persistent pain in migraine sufferers and non-migraine sufferers, and treatment with anti-migraine drugs normalizes CGRP activity.

Possible CGRP involvement in migraine has been the basis for the development and clinical testing of a number of compounds, including for example, olcegepant (Boehringer Ingelheim, Ridgefield, Conn.), telcagepant (Merck Sharp & Dohme Corp., Kenilworth, N.J.), ubrogepant (Allergan plc, Dublin, Ireland), rimegepant (Biohaven Pharmaceutical Holding Company Ltd., New Haven, Conn.), galcanezumab (Eli Lilly and Company, Indianapolis, Ind.), fremanezumab (Teva Pharmaceutical Industries, Petah Tikva, Israel), eptinezumab (Alder Biopharmaceuticals, Inc., Bothell, Wash.), and erenumab (Amgen Inc., Thousand Oaks, Calif.). Another compound recently studied for treatment of migraine is lasmiditan (Eli Lilly and Company, Indianapolis, Ind.).

Currently, clinicians use a number of pharmacologic agents for the acute treatment of migraine. A study published by the American Headache Society in 2015 concluded that the medications deemed effective for the acute treatment of migraine fell into the following classes: triptans, ergotamine derivatives, non-steroidal anti-inflammatory drugs (“NSAIDs”), opioids and combination medications. The current standard of care for the acute treatment of migraine is prescription of triptans, which are serotonin 5-HT_{1B/1D} receptor agonists. Triptans have been developed and approved for the acute treatment of migraine over the past two decades. The initial introduction of triptans represented a shift toward drugs more selectively targeting the suspected pathophysiology of migraine. While triptans account for almost 80% of anti-migraine therapies prescribed at office visits by healthcare providers, issues such as an incomplete effect or headache recurrence remain important clinical limitations. In fact, only about 30% of patients from clinical trials are pain free at two hours after taking triptans. In addition, triptans are contraindicated in patients with cardiovascular disease, cerebrovascular disease, or significant risk factors for either because of potential systemic and cerebrovascular vasoconstriction from the 5-HT_{1B}-mediated effects. Also, according to a January 2017 study published in the journal Headache, an estimated 2.6 million migraine sufferers in the United States have a cardiovascular event, condition or procedure that limits the potential of triptans as a treatment option.

Accordingly, there remains a significant unmet medical need for the treatment of migraine that may provide enhanced patient benefits compared to existing therapies. In addition, CGRP receptor antagonists may be useful pharmacological agents for disorders that involve other CGRP disorders. In addition to migraine, such disorders may include cluster headache (Doods (2001) Curr. Opin. Invest. Drugs 2, 1261-1268; Edvinsson et al. (1994) Cephalalgia 14, 320-327); chronic tension type headache (Ashina et al. (2000) Neurology 14, 1335-1340); pain (Yu et al. (1998) Eur. J Pharmacol. 347, 275-282); chronic pain (Hulsebosch et al. (2000) Pain 86, 163-175); neurogenic inflammation and inflammatory pain (Holzer (1988) Neuroscience 24, 739-768; Delay-Goyet et al. (1992) Acta Physiol. Scanda. 146, 537-538; Salmon et al. (2001) Nature Neurosci. 4, 357-358); eye pain (May et al. (2002) Cephalalgia 22, 195-196), tooth pain (Awawdeh et al. (2002) Int. Endocrin. J35, 30-36), non-insulin dependent diabetes mellitus (Molina etal. (1990) Diabetes 39, 260- 265); vascular disorders; inflammation (Zhang et al. (2001) Pain 89,265); arthritis, bronchial hyperreactivity, asthma, (Foster et al. (1992) Ann. NY Acad. Sci. 657, 397-404; Schini et al. (1994) Am. J Physiol. 267, H2483-H2490; Zheng et al. (1993) J Viral. 67, 5786-5791); shock, sepsis (Beer et al. (2002) Crit. Care Med. 30, 1794-1798); opiate withdrawal syndrome (Salmon et al. (2001) Nature Neurosci. 4, 357-358); morphine tolerance (Menard et al. (1996) J Neurosci. 16, 2342-2351); hot flashes in men and women (Chen et al. (1993) Lancet 342, 49; Spetz et al. (2001) J Urology 166, 1720-1723); allergic dermatitis (Wallengren (2000) Contact Dermatitis 43, 137-143); psoriasis; encephalitis, brain trauma, ischaemia, stroke, epilepsy, and neurodegenerative diseases (Rohrenbeck et al. (1999) Neurobia Dis. 6, 15-34); skin diseases (Geppetti and Holzer, Eds., Neurogenic Inflammation, 1996, CRC Press, Boca Raton, Fla.), neurogenic cutaneous redness, skin rosaceousness and erythema; tinnitus (Herzog et al. (2002) J Membr. Biol. 189,225); obesity (Walker et al. (2010) Endocrinology 151, 4257-4269); inflammatory bowel disease, irritable bowel syndrome, (Hoffman et al. (2002) Scand. J Gastroenterol. 37, 414-422) and cystitis.

### SUMMARY OF THE INVENTION

The present invention is directed to the treatment of CGRP related conditions, e.g., migraine, by orally administering to the patient a pharmaceutical composition including a pharmaceutically acceptable carrier and a therapeutically effective amount of rimegepant, or a pharmaceutically acceptable salt thereof

In an embodiment, the pharmaceutical composition may be a fast-dispersing pharmaceutical composition.

In an embodiment, administering the pharmaceutical composition may result in migraine pain freedom within 2 hours of the time of administration.

In an embodiment, administering the pharmaceutical composition may result in at least 21.2% of patients achieving migraine pain freedom within 2 hours of the time of administration.

In an embodiment, administering the pharmaceutical composition may result in freedom from most bothersome symptoms within 2 hours of the time of administration.

In an embodiment, administering the pharmaceutical composition may result in at least 35.1% of patients achieving freedom from most bothersome symptoms within 2 hours of the time of administration.

In an embodiment, the most bothersome symptoms may be photophobia, nausea, phonophobia, or a combination thereof.

In an embodiment, administering the pharmaceutical composition may result in pain relief within 90 minutes of the time of administration.

In an embodiment, administering the pharmaceutical composition may result in pain relief within 2 hours of the time of administration.

In an embodiment, administering the pharmaceutical composition may result in at least 59.3% of patients achieving pain relief within 2 hours of the time of administration.

In an embodiment, administering the pharmaceutical composition may result in sustained pain relief within 2 to 48 hours of the time of administration.

In an embodiment, administering the pharmaceutical composition may result in at least 42.2% of patients achieving sustained pain relief within 2 to 48 hours of the time of administration.

In an embodiment, administering the pharmaceutical composition may result in sustained pain freedom within 2 to 48 hours of the time of administration.

In an embodiment, administering the pharmaceutical composition may result in at least 13.5% of patients achieving sustained pain freedom within 2 to 24 hours of the time of administration.

In an embodiment, administering the pharmaceutical composition may result in reduced usage of rescue medication within 24 hours of the time of administration.

In an embodiment, administering the pharmaceutical composition may result in at most 14.2% of patients using rescue medication within 24 hours of the time of administration.

In an embodiment, administering the pharmaceutical composition may result in normal function within 2 hours of the time of administration.

In an embodiment, administering the pharmaceutical composition may result in at least 38.1% of patients reporting normal function within 2 hours of the time of administration.

In an embodiment, the daily dose of rimegepant may be about 75 mg.

In an embodiment, the pharmaceutical composition may be administered to the patient up to 15 times in a 30-day period.

In an embodiment, the pharmaceutically acceptable salt or rimegepant may be rimegepant hemisulfate. The daily dose of rimegepant hemisulfate may be about 85.65 mg.

In an embodiment, the pharmaceutical composition may further include benzyl alcohol, eucalyptol, gelatin, limonene, mannitol, menthol, menthone, menthyl acetate, sucralose, vanillin, or a combination thereof.

In an embodiment, 1.3% or less of patients who received the pharmaceutical composition may experience nausea.

In an embodiment, 0.15% or less of patients who received the pharmaceutical composition may experience hypersensitivity.

In an embodiment, the hypersensitivity may include dyspnea, rash, or a combination thereof.

### BRIEF DESCRIPTION OF THE DRAWINGS

These and/or other aspects will become apparent and more readily appreciated from the following description of the embodiments, taken in conjunction with the accompanying drawings in which:

FIG. 1 is a graph of percent achieving pain freedom versus time in hours from dose, which illustrates percentage of patients achieving pain freedom within 2 hours in Study 1; and

FIG. 2 is a graph of percent achieving most bothersome syndrome (MBS) freedom versus time in hours from dose, which illustrates additional migraine efficacy endpoints in Study 1.

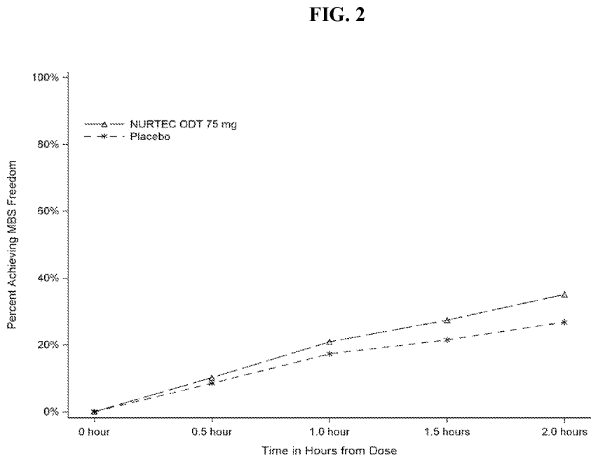

### DETAILED DESCRIPTION OF THE INVENTION

The following detailed description is provided to aid those skilled in the art in practicing the present invention. Those of ordinary skill in the art may make modifications and variations in the embodiments described herein without departing from the spirit or scope of the present disclosure. Unless otherwise defined, all technical and scientific terms used herein have the same meaning as commonly understood by one of ordinary skill in the art to which this disclosure belongs. The terminology used in the description is for describing particular embodiments only and is not intended to be limiting.

As used in this application, except as otherwise expressly provided herein, each of the following terms shall have the meaning set forth below. Additional definitions are set forth throughout the application. In instances where a term is not specifically defined herein, that term is given an art-recognized meaning by those of ordinary skill applying that term in context to its use in describing the present invention.

The articles “a” and “an” refer to one or to more than one (i.e., to at least one) of the grammatical object of the article unless the context clearly indicates otherwise. By way of example, “an element” means one element or more than one element.

The term “about” refers to a value or composition that is within an acceptable error range for the particular value or composition as determined by one of ordinary skill in the art, which will depend in part on how the value or composition is measured or determined, i.e., the limitations of the measurement system. For example, “about” can mean within 1 or more than 1 standard deviation per the practice in the art. Alternatively, “about” can mean a range of up to 1%, 5%, 10% or 20% (i.e., ±10% or ±20%) depending on the context of the application. For example, about 3 mg can include any number between 2.7 mg and 3.3 mg (for 10%) or between 2.4 mg and 3.6 mg (for 20%). Furthermore, particularly with respect to biological systems or processes, the terms can mean up to an order of magnitude or up to 5-fold of a value. When particular values or compositions are provided in the application and claims, unless otherwise stated, the meaning of “about” should be assumed to be within an acceptable error range for that particular value or composition.

The term “administering” refers to the physical introduction of a composition comprising a therapeutic agent to a subject, using any of the various methods and delivery systems known to those skilled in the art. Administering can also be performed, for example, once, a plurality of times, and/or over one or more extended periods and can be a therapeutically effective dose or a subtherapeutic dose.

The term “AUC” (area under the curve) refers to a total amount of drug absorbed or exposed to a subject. Generally, AUC may be obtained from mathematical method in a plot of drug concentration in the subject over time until the concentration is negligible. The term “AUC” could also refer to partial AUC at specified time intervals.

The term “Cₘₐₓ” refers to a maximum concentration of a drug in blood, serum, a specified compartment or test area of a subject between administration of a first dose and administration of a second dose. The term Cₘₐₓ could also refer to dose normalized ratios, if specified.

The terms “in combination with” and “in conjunction with” refer to administration of one treatment modality in addition to another treatment modality. As such, “in combination with” or “in conjunction with” refers to administration of one treatment modality before, during, or after administration of the other treatment modality to the subject.

The term “pharmaceutically acceptable salt” refers to a salt form of one or more of the compounds described herein which are typically presented to increase the solubility of the compound in the gastric or gastroenteric juices of the patient's gastrointestinal tract in order to promote dissolution and the bioavailability of the compounds. Pharmaceutically acceptable salts include those derived from pharmaceutically acceptable inorganic or organic bases and acids, where applicable. Suitable salts include, for example, those derived from alkali metals such as potassium and sodium, alkaline earth metals such as calcium, magnesium and ammonium salts, among numerous other acids and bases well known in the pharmaceutical art.

The terms “subject” and “patient” refer any human or nonhuman animal. The term “nonhuman animal” includes, but is not limited to, vertebrates such as nonhuman primates, sheep, dogs, and rodents such as mice, rats and guinea pigs. In some embodiments, the subject is a human. The terms, “subject” and “patient” are used interchangeably herein.

The terms “effective amount”, “therapeutically effective amount”, “therapeutically effective dosage” and “therapeutically effective dose” of an agent (also sometimes referred to herein as a “drug”) refers to any amount of the agent that, when used alone or in combination with another agent, protects a subject against the onset of a disease or promotes disease regression evidenced by a decrease in severity of disease symptoms, an increase in frequency and duration of disease symptom-free periods, or relief from impairment or disability due to the disease affliction. The therapeutically effective amount of an agent can be evaluated using a variety of methods known to the skilled practitioner, such as in human subjects during clinical trials, in animal model systems predictive of efficacy in humans, or by assaying the activity of the agent in in vitro assays.

The term “Tₘₐₓ” refers to a time or period after administration of a drug when the maximum concentration (Cₘₐₓ) is reached in blood, serum, a specified compartment or test area of a subject.

The term “treatment” refers to any treatment of a condition or disease in a subject and may include: (i) preventing the disease or condition from occurring in the subject which may be predisposed to the disease but has not yet been diagnosed as having it; (ii) inhibiting the disease or condition, i.e., arresting its development; relieving the disease or condition, i.e., causing regression of the condition; or (iii) ameliorating or relieving the conditions caused by the disease, i.e., symptoms of the disease. Treatment could be used in combination with other standard therapies or alone. Treatment or “therapy” of a subject also includes any type of intervention or process performed on, or the administration of an agent to, the subject with the objective of reversing, alleviating, ameliorating, inhibiting, slowing down or preventing the onset, progression, development, severity or recurrence of a symptom, complication or condition, or biochemical indicia associated with a disease.

With respect to headache, “treatment” is an approach for obtaining beneficial or desired results with a subject. For purposes of this invention, beneficial or desired clinical results include, but are not limited to, one or more of the following: improvement in any aspect of a headache including lessening severity, alleviation of pain intensity, and other associated symptoms, reducing frequency of recurrence, increasing the quality of life of those suffering from the headache, decreasing dose of other medications required to treat the headache and reducing the number of headache days per month. For migraine, other associated symptoms include, but are not limited to, nausea, vomiting, and sensitivity to light, sound, and/or movement. For cluster headache, other associated symptoms include, but are not limited to swelling under or around the eyes, excessive tears, red eye, Rhinorrhea or nasal congestion, and red flushed face.

For purposes of this disclosure, reference is made to the publication by the U.S. Food and Drug Administration (FDA), Guidance for Industry, “Migraine: Developing Drugs for Acute Treatment”, February 2018, available from https://www.fda.gov/downloads/drugs/guidances/ucm419465.pdf. Terms used in the Examples, such as, for example, most bothersome symptoms (MBS) and Pain Freedom, are described in the FDA Guidance.

The starting materials useful for making the pharmaceutical compositions of the present invention are readily commercially available or can be prepared by those skilled in the art.

The invention encompasses oral fast-dispersing dosage form of rimegepant. The invention further encompasses methods for modulating CGRP and treating patients with medical conditions associated with aberrant levels of CGRP or CGRP receptor signaling by orally administering the compositions. Rimegepant is described chemically as (5S,6S,9R)-5-amino-6-(2,3-difluorophenyl)-6,7,8,9-tetrahydro-5H-cyclohepta[b]pyridin-9-yl 4-(2-oxo-2,3-dihydro-1H-imidazo[4,5-b]pyridin-1-yl)-1-piperidinecarboxylate, and its structural formula is:

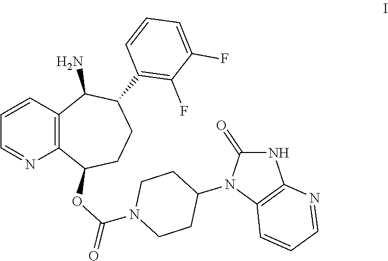 [SMILES: N[C@@H]1c2cccnc2[C@H](OC(=O)N2CCC(n3c(=O)[nH]c4ncccc43)CC2)CC[C@H]1c1cccc(F)c1F]

Rimegepant is described, for example in WO 2011/046997 published Apr. 21, 2011.

The phrase “fast-dispersing dosage form” as used herein refers to compositions which disintegrate or disperse within 1 to 60 seconds, preferably 1 to 30 seconds, more preferably 1 to 10 seconds and particularly 2 to 8 seconds, after being placed in contact with a fluid. The fluid is preferably that found in the oral cavity, i.e., saliva, as with oral administration.

In a preferred embodiment, the compositions of the invention are solid fast-dispersing dosage forms comprising a solid network of the active ingredient, rimegepant, and a water-soluble or water-dispersible carrier containing fish gelatin. Accordingly, the carrier is inert towards the active ingredient. The network is obtained by subliming solvent from a composition in the solid state, the composition comprising the active ingredient and a solution of the carrier in the solvent. The dosage forms according to the invention can be prepared according to the process disclosed in Gregory et al., U.K. Patent No. 1,548,022 using fish gelatin as the carrier. Accordingly, an initial composition (or admixture) comprising the active ingredient and a solution of the fish gelatin carrier in a solvent is prepared followed by sublimation. The sublimation is preferably carried out by freeze drying the composition. The composition can be contained in a mold during the freeze-drying process to produce a solid form in any desired shape. The mold can be cooled using liquid nitrogen or solid carbon dioxide in a preliminary step prior to the deposition of the composition therein. After freezing the mold and composition, they are next subjected to reduced pressure and, if desired, controlled application of heat to aid in sublimation of solvent. The reduced pressure applied in the process can be below about 4 mm Hg, preferably below about 0.3 mm Hg. The freeze dried compositions can then be removed from the mold if desired or stored therein until later use.

When the process is used with active ingredients and fish gelatin as the carrier, a solid fast-dispersing dosage form is produced having the advantages associated with the use of fish gelatin described herein. Generally, fish gelatin is categorized as being from cold water and warm water fish sources and as being of the gelling or non-gelling variety. The non-gelling variety of fish gelatin, in comparison to gelling fish gelatin and bovine gelatin, contains lower proline and hydroxyproline amino acid content, which are known to be associated with cross-linking properties and gelling ability. Non-gelling fish gelatin can remain at solution concentrations of up to about 40% as well as in temperatures as low as 20° C. In an aspect of the invention, the fish gelatin used in accordance with the invention is preferably obtained from cold water fish sources and is the non-gelling type of fish gelatin. More preferably, in one aspect of the invention, the non-hydrolyzed form of non-gelling fish gelatin is used. In an alternative embodiment, spray-dried non-hydrolyzed non-gelling fish gelatin can be used. Fish gelatins suitable for use in the invention are commercially available.

The compositions according to the invention can also contain, in addition to the active ingredient arid fish gelatin carrier, other matrix forming agents and secondary components. Matrix forming agents suitable for use in the present invention include materials derived from animal or vegetable proteins, such as other gelatins, dextrins and soy, wheat and psyllium seed proteins; gums such as acacia, guar, agar, and xanthan; polysaccharides; alginates; carboxymethylcelluloses; carrageenans; dextrans; pectins; synthetic polymers such as polyvinylpyrrolidone; and polypeptide/protein or polysaccharide complexes such as gelatin-acacia complexes.

Other materials which may also be incorporated into the fast-dissolving compositions of the present invention include sugars such as mannitol, dextrose, lactose, galactose, and trehalose; cyclic sugars such as cyclodextrin; inorganic salts such as sodium phosphate, sodium chloride and aluminum silicates; and amino acids having from 2 to 12 carbon atoms such as glycine, L-alanine, L-aspartic acid, L-glutamic acid, L-hydroxyproline, L-isoleucine, L-leucine and L-phenylalanine. One or more matrix forming agents may be incorporated into the solution or suspension prior to solidification (freezing). The matrix forming agent may be present in addition to a surfactant or to the exclusion of a surfactant. In addition to forming the matrix, the matrix forming agent may aid in maintaining the dispersion of any active ingredient within the solution of suspension. This is especially helpful in the case of active agents that are not sufficiently soluble in water and must, therefore, be suspended rather than dissolved. Secondary components such as preservatives, antioxidants, surfactants, viscosity enhancers, coloring agents, flavoring agents, pH modifiers, sweeteners or taste-masking agents may also be incorporated into the fast-dissolving compositions. Suitable coloring agents include red, black and yellow iron oxides and FD & C dyes such as FD&C Blue No. 2 and FD&C Red No. 40 available from Ellis & Everard. Suitable flavoring agents include mint, raspberry, licorice, orange, lemon, grapefruit, caramel, vanilla, cherry and grape flavors and combinations of these. Suitable pH modifiers include the edible acids and bases, such as citric acid, tartaric acid, phosphoric acid, hydrochloric acid, maleic acid and sodium hydroxide. Suitable sweeteners include, for example, sucralose, aspartame, acesulfame K and thaumatin. Suitable taste-masking agents include, for example, sodium bicarbonate, ion exchange resins, cyclodextrin inclusion compounds, adsorbates or microencapsulated actives.

A typical route of administering the fast-dispersing dosage form of the present invention is oral. Pharmaceutical compositions according to certain embodiments of the present invention are formulated so as to allow the active ingredients contained therein to be bioavailable upon administration of the composition to a patient. Compositions that will be administered to a subject or patient may take the form of one or more dosage units. Actual methods of preparing such dosage forms are known, or will be apparent, to those skilled in this art; for example, see Remington: The Science and Practice of Pharmacy, 20th Edition (Philadelphia College of Pharmacy and Science, 2000).

Solid compositions are normally formulated in dosage units providing from about 1 to about 1000 mg of the active ingredient per dose. Some examples of solid dosage units are 0.1 mg, 1 mg, 10 mg, 37.5 mg, 75 mg, 100 mg, 150 mg, 300 mg, 500 mg, 600 mg and 1000 mg. Typical dose ranges in accordance with the present invention include from about 10-600 mg, 25-300 mg, 25-150 mg, 50-100 mg, 60-90 mg, and 70-80 mg. Liquid compositions are generally in a unit dosage range of 1-100 mg/mL. Some examples of liquid dosage units are 0.1 mg/mL, 1 mg/mL, 10 mg/mL, 25 mg/mL, 50 mg/mL, and 100 mg/mL.

The pharmaceutical compositions of the present invention can be prepared in any suitable dosage form including, for example, such as tablets, capsules, nasal sprays, powders, granules, ointments, solutions, suppositories, injections, inhalants, gels, microspheres, and aerosols.

The pharmaceutical compositions of the present invention containing rimegepant typically also include other pharmaceutically acceptable carriers (also referred to as excipients) such as, for example, binders, lubricants, diluents, coatings, disintegrants, barrier layer components, glidants, coloring agents, solubility enhancers, gelling agents, fillers, proteins, co-factors, emulsifiers, solubilizing agents, suspending agents, flavorants, preservatives and mixtures thereof. The choice of excipients depends on the desired characteristics of the compositions and on the nature of other pharmacologically active compounds in the formulation.

Suitable excipients are known to those skilled in the art (see Handbook of Pharmaceutical Excipients, fifth edition, 2005 edited by Rowe et al., McGraw Hill).

Examples of pharmaceutically acceptable carriers that may be used in preparing the pharmaceutical compositions of the present invention may include, but are not limited to, fillers such as sugars, including lactose, sucrose, mannitol, or sorbitol; cellulose preparations such as maize starch, wheat starch, rice starch, potato starch, gelatin, gum tragacanth, methyl cellulose, hydroxypropyl methyl-cellulose, sodium carboxymethylcellulose, polyvinyl-pyrrolidone (PVP), talc, calcium sulphate, vegetable oils, synthetic oils, polyols, alginic acid, phosphate buffered solutions, emulsifiers, isotonic saline, pyrogen-free water and combinations thereof. If desired, disintegrating agents may be combined as well, and exemplary disintegrating agents may be, but not limited to, cross-linked polyvinyl pyrrolidone, agar, or alginic acid or a salt thereof such as sodium alginate. In an aspect of the invention, the flavoring agent is selected from mint, peppermint, berries, cherries, menthol and sodium chloride flavoring agents, and combinations thereof. In an aspect of the invention, the sweetener is selected from sugar, sucralose, aspartame, acesulfame, neotame, and combinations thereof.

In general, the pharmaceutical compositions of the present invention may be manufactured in conventional methods known in the art, for example, by means of conventional mixing, dissolving, granulating, dragee-making, levigating, emulsifying, encapsulating, entrapping, lyophilizing processes and the like.

In an aspect of the invention the pharmaceutical compositions are prepared in oral solid molded fast-dispersing dosage form, such as described in U.S. Pat. No. 9,192,580, issued Nov. 24, 2015.

In some embodiments, a method may comprise administering to a subject one or more additional agent(s) simultaneously or sequentially with the rimegepant. In some embodiments, an additional agent may be an anti-headache medication such as an example anti-headache medication (e.g., 5-HT₁ agonists, triptans, ergot alkaloids, opiates, adrenergic antagonists, NSAIDs or antibodies) known in the art. In some embodiments, a therapeutic effect may be greater as compared to use of rimegepant or one or more additional agent(s) alone. Accordingly, a synergistic effect between rimegepant and the one or more additional agents may be achieved. In some embodiments, the one or more additional agent(s) may be taken by a subject prophylactically.

In addition to migraine, other CGRP related disorders that may be treated by the pharmaceutical compositions and methods of the present invention include, for example, cluster headache; chronic tension type headache; chronic pain; neurogenic inflammation and inflammatory pain; eye pain; tooth pain; non-insulin dependent diabetes mellitus; vascular disorders; inflammation; arthritis; bronchial hyperreactivity; asthma; shock; sepsis; opiate withdrawal syndrome; morphine tolerance; hot flashes in men and women; allergic dermatitis; psoriasis; encephalitis, brain trauma, ischaemia, stroke, epilepsy, and neurodegenerative diseases; skin diseases; neurogenic cutaneous redness, skin rosaceousness and erythema; tinnitus; obesity; inflammatory bowel disease; irritable bowel syndrome; and cystitis.

In an aspect, the invention also provides kits for use in the instant methods. Kits can include one or more containers comprising a pharmaceutical composition described herein and instructions for use in accordance with any of the methods described herein. Generally, these instructions comprise a description of administration of the pharmaceutical composition to treat, ameliorate or prevent headache (such as migraine), or other CRGP disorder, according to any of the methods described herein. The kit may, for example, comprise a description of selecting an individual suitable for treatment based on identifying whether that individual has headache or whether the individual is at risk of having headache. The instructions are typically provided in the form of a package insert, or label, in accordance with the requirements of the regulatory having authority over the jurisdiction where the pharmaceutical composition is to be provided to patients.

In accordance with the present invention, administration of the pharmaceutical compositions comprising rimegepant to a subject may promote a reduction in severity (which can include reducing need for and/or amount of (e.g., exposure to) other drugs and/or therapies generally used for this condition, including, for example, ergotamine, dihydroergotamine, or triptans for migraine), duration, and/or frequency (including, for example, delaying or increasing time to next episodic attack in an individual).

In addition, administration of the pharmaceutical compositions comprising rimegepant to a subject may promote a lessening or improvement of one or more symptoms of headache, or a reduction in the duration of a symptom, as compared to not administering a treatment.

In addition, administration of the pharmaceutical compositions comprising rimegepant to a subject may promote a reduction in the frequency of headache attacks in an individual (as compared to the level before treatment) in a certain time period, e.g., per month. For example, the frequency of attacks may be reduced by at least about any of 10%, 15%, 20%, 25%, 30%, 35%, 40%, 45%, 50%, 55%, 60%, 65%, or 70% in the individual as compared to the level before treatment.

In addition, administration of the pharmaceutical compositions comprising rimegepant to a subject may promote a delay in the development of headache, i.e., to defer, hinder, slow, retard, stabilize, and/or postpone progression of the disease. This delay can be of varying lengths of time, depending on the history of the disease and/or individuals being treated.

In addition, administration of the pharmaceutical compositions comprising rimegepant to a subject may delay the development or progression of a headache, i.e., delay of the initial manifestations and/or ensuing progression of the disorder. Development of headache can be detectable and assessed using standard clinical techniques as well known in the art. However, development also refers to progression that may be undetectable.

In addition, administration of the pharmaceutical compositions comprising rimegepant may result in increased number of patients achieving migraine pain freedom within 2 hours of the time of administration. For example, the number of patients achieving migraine pain freedom within 2 hours of the time of administration may be about 5% or greater, about 10% or greater, about 15% or greater, about 20% or greater, about 25% or greater, about 30% or greater, about 35% or greater, about 40% or greater, about 45% or greater, about 50% or greater, about 55% or greater, about 60% or greater, about 65% or greater, about 70% or greater, about 75% or greater, about 80% or greater, about 85% or greater, about 90% or greater, about 95% or greater, or about 100%. For example, the number of patients achieving migraine pain freedom within 2 hours of the time of administration may be about 21.2%.

Administration of the pharmaceutical composition may result in migraine pain freedom within 2 hours of the time of administration. For example, administration of the pharmaceutical composition may result in migraine pain freedom within 1.9 hours, within 1.8 hours, within 1.7 hours, within 1.6 hours, within 1.5 hours, within 1.4 hours, within 1.3 hours, within 1.2 hours, within 1.1 hours, within 1.0 hours, within 0.9 hours, within 0.8 hours, within 0.7 hours, within 0.6 hours, within 0.5 hours, within 0.4 hours, within 0.3 hours, within 0.2 hours, or within 0.1 hours of the time of administration.

In addition, administration of the pharmaceutical compositions comprising rimegepant may result in increased number of patients achieving freedom from most bothersome symptoms within 2 hours of the time of administration. For example, the number of patients achieving freedom from most bothersome symptoms within 2 hours of the time of administration may be about 5% or greater, about 10% or greater, about 15% or greater, about 20% or greater, about 25% or greater, about 30% or greater, about 35% or greater, about 40% or greater, about 45% or greater, about 50% or greater, about 55% or greater, about 60% or greater, about 65% or greater, about 70% or greater, about 75% or greater, about 80% or greater, about 85% or greater, about 90% or greater, about 95% or greater, or about 100%. For example, the number of patients achieving migraine pain freedom within 2 hours of the time of administration may be about 35.1%. The most bothersome symptoms may include photophobia, nausea, phonophobia, or a combination thereof

Administration of the pharmaceutical composition may result in freedom from most bothersome symptoms within 2 hours of the time of administration. For example, administration of the pharmaceutical composition may result in freedom from most bothersome symptoms within 1.9 hours, within 1.8 hours, within 1.7 hours, within 1.6 hours, within 1.5 hours, within 1.4 hours, within 1.3 hours, within 1.2 hours, within 1.1 hours, within 1.0 hours, within 0.9 hours, within 0.8 hours, within 0.7 hours, within 0.6 hours, within 0.5 hours, within 0.4 hours, within 0.3 hours, within 0.2 hours, or within 0.1 hours of the time of administration.

In addition, administration of the pharmaceutical compositions comprising rimegepant may result in increased number of patients achieving pain relief within 90 minutes of the time of administration. For example, the number of patients achieving pain relief within 90 minutes of the time of administration may be about 5% or greater, about 10% or greater, about 15% or greater, about 20% or greater, about 25% or greater, about 30% or greater, about 35% or greater, about 40% or greater, about 45% or greater, about 50% or greater, about 55% or greater, about 60% or greater, about 65% or greater, about 70% or greater, about 75% or greater, about 80% or greater, about 85% or greater, about 90% or greater, about 95% or greater, or about 100%. For example, the number of patients achieving pain relief within 90 minutes of the time of administration may be about 49.6%.

Administration of the pharmaceutical composition may result in achieving pain relief within 90 minutes of the time of administration. For example, administration of the pharmaceutical composition may result in achieving pain relief within 85 minutes, within 80 minutes, within 75 minutes, within 70 minutes, within 65 minutes, within 60 minutes, within 55 minutes, within 50 minutes, within 45 minutes, within 40 minutes, within 35 minutes, within 30 minutes, within 25 minutes, within 20 minutes, within 15 minutes, within 10 minutes, or within 5 minutes of the time of administration.

In addition, administration of the pharmaceutical compositions comprising rimegepant may result in increased number of patients achieving pain relief within 2 hours of the time of administration. For example, the number of patients achieving pain relief within 2 hours of the time of administration may be about 5% or greater, about 10% or greater, about 15% or greater, about 20% or greater, about 25% or greater, about 30% or greater, about 35% or greater, about 40% or greater, about 45% or greater, about 50% or greater, about 55% or greater, about 60% or greater, about 65% or greater, about 70% or greater, about 75% or greater, about 80% or greater, about 85% or greater, about 90% or greater, about 95% or greater, or about 100%. For example, the number of patients achieving pain relief within 2 hours of the time of administration may be about 59.3%.

Administration of the pharmaceutical composition may result in achieving pain relief within 2 hours of the time of administration. For example, administration of the pharmaceutical composition may result in achieving migraine pain relief within 1.9 hours, within 1.8 hours, within 1.7 hours, within 1.6 hours, within 1.5 hours, within 1.4 hours, within 1.3 hours, within 1.2 hours, within 1.1 hours, within 1.0 hours, within 0.9 hours, within 0.8 hours, within 0.7 hours, within 0.6 hours, within 0.5 hours, within 0.4 hours, within 0.3 hours, within 0.2 hours, or within 0.1 hours of the time of administration.

In addition, administration of the pharmaceutical compositions comprising rimegepant may result in increased number of patients achieving sustained pain relief within 2 to 48 hours of the time of administration. For example, the number of patients achieving sustained pain relief within 2 to 48 hours of the time of administration may be about 5% or greater, about 10% or greater, about 15% or greater, about 20% or greater, about 25% or greater, about 30% or greater, about 35% or greater, about 40% or greater, about 45% or greater, about 50% or greater, about 55% or greater, about 60% or greater, about 65% or greater, about 70% or greater, about 75% or greater, about 80% or greater, about 85% or greater, about 90% or greater, about 95% or greater, or about 100%. For example, the number of patients achieving sustained pain relief within 2 to 48 hours of the time of administration may be about 42.2%.

In addition, administration of the pharmaceutical compositions comprising rimegepant may result in increased number of patients achieving sustained pain freedom within 2 to 24 hours of the time of administration. For example, the number of patients achieving sustained pain freedom within 2 to 24 hours of the time of administration may be about 5% or greater, about 10% or greater, about 15% or greater, about 20% or greater, about 25% or greater, about 30% or greater, about 35% or greater, about 40% or greater, about 45% or greater, about 50% or greater, about 55% or greater, about 60% or greater, about 65% or greater, about 70% or greater, about 75% or greater, about 80% or greater, about 85% or greater, about 90% or greater, about 95% or greater, or about 100%. For example, the number of patients achieving sustained pain freedom within 2 to 24 hours of the time of administration may be about 15.7%.

In addition, administration of the pharmaceutical compositions comprising rimegepant may result in increased number of patients achieving sustained pain freedom within 2 to 48 hours of the time of administration. For example, the number of patients achieving sustained pain freedom within 2 to 48 hours of the time of administration may be about 5% or greater, about 10% or greater, about 15% or greater, about 20% or greater, about 25% or greater, about 30% or greater, about 35% or greater, about 40% or greater, about 45% or greater, about 50% or greater, about 55% or greater, about 60% or greater, about 65% or greater, about 70% or greater, about 75% or greater, about 80% or greater, about 85% or greater, about 90% or greater, about 95% or greater, or about 100%. For example, the number of patients achieving sustained pain freedom within 2 to 48 hours of the time of administration may be about 13.5%.

In addition, administration of the pharmaceutical compositions comprising rimegepant may result in reduced number of patients using rescue medication within 24 hours of the time of administration. For example, the number of patients using rescue medication within 24 hours of the time of administration may be about 95% or less, about 90% or less, about 85% or less, about 80% or less, about 75% or less, about 70% or less, about 65% or less, about 60% or less, about 55% or less, about 50% or less, about 45% or less, about 40% or less, about 35% or less, about 30% or less, about 25% or less, about 20% or less, about 15% or less, about 10% or less, or about 5% or less. For example, the number of patients using rescue medication within 24 hours of the time of administration may be about 14.2%.

In addition, administration of the pharmaceutical compositions comprising rimegepant may result in increased number of patients reporting normal function within 2 hours of the time of administration. For example, the number of patients reporting normal function within 2 to 24 hours of the time of administration may be about 5% or greater, about 10% or greater, about 15% or greater, about 20% or greater, about 25% or greater, about 30% or greater, about 35% or greater, about 40% or greater, about 45% or greater, about 50% or greater, about 55% or greater, about 60% or greater, about 65% or greater, about 70% or greater, about 75% or greater, about 80% or greater, about 85% or greater, about 90% or greater, about 95% or greater, or about 100%. For example, the number of patients reporting normal function within 2 hours of the time of administration may be about 38.1%.

Administration of the pharmaceutical composition may result in reporting normal function within 2 hours of the time of administration. For example, administration of the pharmaceutical composition may result in reporting normal function within 1.9 hours, within 1.8 hours, within 1.7 hours, within 1.6 hours, within 1.5 hours, within 1.4 hours, within 1.3 hours, within 1.2 hours, within 1.1 hours, within 1.0 hours, within 0.9 hours, within 0.8 hours, within 0.7 hours, within 0.6 hours, within 0.5 hours, within 0.4 hours, within 0.3 hours, within 0.2 hours, or within 0.1 hours of the time of administration.

A patient may have 15 migraines or less in a 30-day period. For example, the patient may have 15 migraines, 14 migraines, 13 migraines, 12 migraines, 11 migraines, 10 migraines, 9 migraines, 8 migraines, 7 migraines, 6 migraines, 5 migraines, 4 migraines, 3 migraines, 2 migraines, or 1 migraine in a 30-day period.

The pharmaceutical composition may be administered to the patient up to 15 times in a 30-day period. For example, the pharmaceutical composition may be administered to the patient once, twice, 3 times, 4 times, 5 times, 6 times, 7 times, 8 times, 9 times, 10 times, 11 times, 12 times, 13 times, 14 times, or 15 times in a 30-day period.

In an embodiment, the oral fast-dispersing dosage form may contain rimegepant hemisulfate, which is described chemically as (5S,6S,9R)-5-amino-6-(2,3-difluorophenyl)-6,7,8,9-tetrahydro-5H-cyclohepta[b]pyridin-9-yl 4-(2-oxo-2,3-dihydro-1H-imidazo[4,5-b]pyridin-1-yl)-1-piperidinecarboxylate hemisulfate sesquihydrate and its structural formula is:

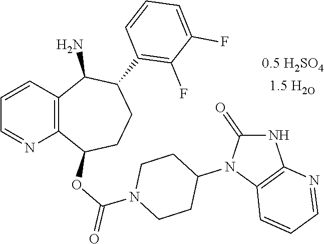 [SMILES: N[C@@H]1c2cccnc2[C@H](OC(=O)N2CCC(n3c(=O)[nH]c4ncccc43)CC2)CC[C@H]1c1cccc(F)c1F]

The empirical formula of rimegepant hemisulfate is C₂₈H₂₈F₂N₆O₃ 0.5 H₂SO₄ 1.5 H₂O, representing a molecular weight of 610.63. Rimegepant free base has a molecular weight of 534.56. The daily dose of rimegepant hemisulfate may be about 85.65 mg.

Generally, rimegepant may be included in the composition of the present invention in all pharmaceutically acceptable salt forms. Pharmaceutically acceptable salts are those in which the counter ions do not contribute significantly to the physiological activity or toxicity of the compounds and as such function as pharmacological equivalents. These salts can be made according to common organic techniques employing commercially available reagents. Some anionic salt forms include acetate, acistrate, besylate, bromide, chloride, citrate, fumarate, glucouronate, hydrobromide, hydrochloride, hydroiodide, iodide, lactate, maleate, mesylate, nitrate, pamoate, phosphate, succinate, sulfate, tartrate, tosylate, and xinofoate, but are not limited thereto. Some cationic salt forms include ammonium, aluminum, benzathine, bismuth, calcium, choline, diethylamine, diethanolamine, lithium, magnesium, meglumine, 4-phenylcyclohexylamine, piperazine, potassium, sodium, tromethamine, and zinc, but are not limited thereto.

The invention is intended to include all isotopes of atoms occurring in the compound having formula I. Isotopes include those atoms having the same atomic number but different mass numbers. By way of general example and without limitation, isotopes of hydrogen include deuterium and tritium. Isotopes of carbon include ¹³C and ¹⁴C. Isotopically-labeled compounds of the invention can generally be prepared by conventional techniques known to those skilled in the art or by processes analogous to those described herein, using an appropriate isotopically-labeled reagent in place of the non-labeled reagent otherwise employed. Such compounds may have a variety of potential uses, for example as standards and reagents in determining biological activity. In the case of stable isotopes, such compounds may have the potential to favorably modify biological, pharmacological, or pharmacokinetic properties.

The therapeutically active component may include two or more compounds, each of which may be orally bioavailable active pharmaceutical ingredient (“API”), for example, an anti-migraine drug.

The invention is further illustrated by the following non-limiting examples.

### EXAMPLES

The present invention is exemplified with rimegepant hemisulfate, which is a white to off-white, crystalline solid that is slightly soluble in water. The oral fast-dispersing dosage form containing rimegepant hemisulfate is hereinafter referred to as “NURTEC ODT.”

NURTEC ODT may be an orally disintegrating tablet for sublingual or oral use, and may contain 85.65 mg rimegepant hemisulfate, equivalent to 75 mg rimegepant free base. NURTEC ODT may further include the following inactive ingredients: benzyl alcohol, eucalyptol, gelatin, limonene, mannitol, menthol, menthone, menthyl acetate, sucralose, and vanillin.

### Indications and Usage

NURTEC ODT is indicated for the acute treatment of migraine with or without aura in adults. NURTEC ODT is not indicated for the preventive treatment of migraine.

### Dosage and Administration

Dosing Information

The recommended dose of NURTEC ODT is 75 mg taken orally. The maximum dose in a 24-hour period is 75 mg. The safety of treating more than 15 migraines in a 30-day period has not been established.

Administration Information

A patient taking NURTEC ODT should be instructed as follows:
Use dry hands when opening the blister pack.
Peel back the foil covering of one blister and gently remove the orally disintegrating tablet (ODT). Do not push the ODT through the foil.
As soon as the blister is opened, remove the ODT and place on the tongue; alternatively, the ODT may be placed under the tongue.
The ODT will disintegrate in saliva so that it can be swallowed without additional liquid.
Take the ODT immediately after opening the blister pack. Do not store the ODT outside the blister pack for future use.

Concomitant Administration with Strong or Moderate CYP3A4 Inhibitors

Concomitant administration of NURTEC ODT with strong inhibitors of CYP3A4 should be avoided. Also avoided should be another dose of NURTEC ODT within 48 hours when it is concomitantly administered with moderate inhibitors of CYP3A4 [see Drug Interactions and Clinical Pharmacology].

Concomitant Administration with Strong or Moderate CYP3A Inducers

Concomitant administration of NURTEC ODT with strong or moderate inducers of CYP3A which may lead to loss of efficacy of NURTEC ODT should be avoided [see Drug Interactions and Clinical Pharmacology].

Concomitant Administration with Inhibitors of P-gp or BCRP

Avoid concomitant administration of NURTEC ODT with inhibitors of P-glycoprotein (P-gp) or breast cancer resistance protein (BCRP) should be avoided [see Drug Interactions and Clinical Pharmacology).

### Dosage Forms and Strengths

NURTEC ODT may be in the form of orally disintegrating tablets, which may be white to off-white, which may be circular, and which may be debossed with the symbol [symbol] . Each tablet may contain 75 mg of rimegepant.

### Contraindications

NURTEC ODT is contraindicated in patients with a history of hypersensitivity reaction to rimegepant, NURTEC ODT, or any of its components. However, delayed serious hypersensitivity has occurred [see Warnings and Precautions].

### Warnings and Precautions

Hypersensitivity Reactions

Hypersensitivity reactions, including dyspnea and rash, have occurred with NURTEC ODT in clinical studies. Hypersensitivity reactions can occur days after administration, and delayed serious hypersensitivity has occurred. If a hypersensitivity reaction occurs, NURTEC

ODT should be discontinued and appropriate therapy should be initiated [see Contraindications].

### Adverse Reactions

The clinically significant adverse reactions may include hypersensitivity reactions, which are discussed in the Warnings and Precautions.

Clinical Trials Experience

Because clinical trials are conducted under widely varying conditions, adverse reaction rates observed in the clinical trials of a drug cannot be directly compared to rates in the clinical trials of another drug and may not reflect the rates observed in clinical practice.

The safety of NURTEC ODT has been evaluated in a randomized, double-blind, placebo-controlled trial (Study 1) in 682 patients with migraine who received one 75 mg dose of NURTEC ODT [see Clinical Studies]. Approximately 85% were female, 74% were White, 21% were Black, and 17% were Hispanic or Latino. The mean age at study entry was 40 years (range 18-75 years of age).

Long-term safety was assessed in an open-label extension study using a different oral dosage form of rimegepant. That study evaluated 1,798 patients, dosing intermittently for up to one year, including 1,131 patients who were exposed to rimegepant 75 mg for at least 6 months, and 863 who were exposed for at least one year, all of whom treated an average of at least two migraine attacks per month.

The most common adverse reaction in Study 1 was nausea (2% in patients who received NURTEC ODT compared to 0.4% of patients who received placebo).

Hypersensitivity including dyspnea and severe rash, occurred in less than 1% of patients treated with NURTEC ODT [see Contraindications and Warnings and Precautions].

### Drug Interactions

CYP3A4 Inhibitors

Concomitant administration of NURTEC ODT with strong inhibitors of CYP3A4 results in a significant increase in rimegepant exposure. Avoid concomitant administration of NURTEC ODT with strong inhibitors of CYP3A4 [see Clinical Pharmacology].

Concomitant administration of NURTEC ODT with moderate inhibitors of CYP3A4 may result in increased exposure of rimegepant. Avoid another dose of NURTEC ODT within 48 hours when it is concomitantly administered with moderate inhibitors of CYP3A4 [see Clinical Pharmacology].

CYP3A Inducers

Concomitant administration of NURTEC ODT with strong or moderate inducers of CYP3A can result in a significant reduction in rimegepant exposure, which may lead to loss of efficacy of NURTEC ODT. Avoid concomitant administration of NURTEC ODT with strong or moderate inducers of CYP3A [see Clinical Pharmacology].

Transporters

Rimegepant is a substrate of P-gp and BCRP efflux transporters. Concomitant administration of NURTEC ODT with inhibitors of P-gp or BCRP may result in a significant increase in rimegepant exposure [see Clinical Pharmacology]. Avoid NURTEC ODT with inhibitors of P-gp or BCRP.

### Use in Specific Populations

Pregnancy

Risk Summary

There are no adequate data on the developmental risk associated with the use of NURTEC ODT in pregnant women. In animal studies, oral administration of rimegepant during organogenesis resulted in adverse effects on development in rats (decreased fetal body weight and increased incidence of fetal variations) at exposures greater than those used clinically and which were associated with maternal toxicity. The evaluation of developmental effects following oral administration of rimegepant throughout pregnancy and lactation was inadequate (see Data).

In the U.S. general population, the estimated background risk of major birth defects and miscarriage in clinically recognized pregnancies is 2 to 4% and 15 to 20%, respectively. The estimated rate of major birth defects (2.2 to 2.9%) and miscarriage (17%) among deliveries to women with migraine are similar to rates reported in women without migraine.

Clinical Considerations

Disease-Associated Maternal and/or Embryo/Fetal Risk

Published data have suggested that women with migraine may be at increased risk of preeclampsia and gestational hypertension during pregnancy.

Data

Animal Data

Oral administration of rimegepant (0, 10, 60, or 300 mg/kg/day) to pregnant rats during the period of organogenesis resulted in decreased fetal body weight and an increased incidence of fetal variations at the highest dose tested (300 mg/kg/day), which was associated with maternal toxicity. Plasma exposures (AUC) at the no-effect dose (60 mg/kg/day) for adverse effects on embryofetal development were approximately 45 times that in humans at the maximum recommended human dose (MRHD) of 75 mg/day.

Oral administration of rimegepant (0, 10, 25, or 50 mg/kg/day) to pregnant rabbits during the period of organogenesis resulted in no adverse effects on embryofetal development. The highest dose tested (50 mg/kg/day) was associated with plasma exposures (AUC) approximately 10 times that in humans at the MRHD.

The prenatal and postnatal development study in rats, in which rimegepant (0, 10, 25, or 60 mg/kg/day) was orally administered throughout gestation and lactation, was inadequate to assess for adverse effects of rimegepant during these periods of development.

Lactation

There are no data on the presence of rimegepant or its metabolites in human milk, the effects of rimegepant on the breastfed infant, or the effects of rimegepant on milk production.

There are no animal data on the excretion of rimegepant in milk. The developmental and health benefits of breastfeeding should be considered along with the mother's clinical need for NURTEC ODT and any potential adverse effects on the breastfed infant from NURTEC ODT or from the underlying maternal condition.

Pediatric Use

Safety and effectiveness of NURTEC ODT in pediatric patients have not been established.

Geriatric Use

In pharmacokinetic studies, no clinically significant pharmacokinetic differences were observed between elderly and younger subjects. Clinical studies of NURTEC ODT did not include sufficient numbers of patients aged 65 and over to determine whether they respond differently from younger patients.

Hepatic Impairment

No dosage adjustment of NURTEC ODT is required in patients with mild (Child-Pugh A) or moderate (Child-Pugh B) hepatic impairment. Plasma concentrations of rimegepant were significantly higher in subjects with severe (Child-Pugh C) hepatic impairment. Use of NURTEC ODT in patients with severe hepatic impairment should be avoided [see Clinical Pharmacology].

Renal Impairment

No dosage adjustment of NURTEC ODT is required in patients with mild, moderate, or severe renal impairment. NURTEC ODT has not been studied in patients with end-stage renal disease and in patients on dialysis. Use of NURTEC ODT in patients with end-stage renal disease (CLcr<15 mL/min) should be avoided [see Clinical Pharmacology].

### Overdosage

There is limited clinical experience with NURTEC ODT overdosage. Treatment of an overdose of NURTEC ODT should consist of general supportive measures including monitoring of vital signs and observation of the clinical status of the patient. No specific antidote for the treatment of rimegepant overdose is available.

Rimegepant is unlikely to be significantly removed by dialysis because of high serum protein binding [see Clinical Pharmacology].

### Clinical Pharmacology

Mechanism of Action

Rimegepant is a calcitonin gene-related peptide receptor antagonist.

Pharmacodynamics

The relationship between pharmacodynamic activity and the mechanism(s) by which rimegepant exerts its clinical effects is unknown. No clinically relevant differences in resting blood pressure were observed when rimegepant was concomitantly administered with sumatriptan (12 mg subcutaneous, given as two 6 mg doses separated by one hour) compared with sumatriptan alone to healthy volunteers.

Cardiac Electrophysiology.

At a single dose 4 times the recommended dose, rimegepant does not prolong the QT interval to any clinically relevant extent.

Pharmacokinetics

Absorption

Following oral administration of NURTEC ODT, rimegepant is absorbed with the maximum concentration at 1.5 hours. The absolute oral bioavailability of rimegepant is approximately 64%.

Effects of Food

Following administration of NURTEC ODT under fed condition with a high-fat meal, Tₘₐₓ was delayed by 1 hour and resulted in a 42 to 53% reduction in Cₘₐₓ and a 32 to 38% reduction in AUC. NURTEC ODT was administered without regard to food in clinical safety and efficacy studies. The impact of the reduction in rimegepant exposure because of administration with food on its efficacy is unknown.

Distribution

The steady state volume of distribution of rimegepant is 120 L. Plasma protein binding of rimegepant is approximately 96%.

Elimination

Metabolism

Rimegepant is primarily metabolized by CYP3A4 and to a lesser extent by CYP2C9.

Rimegepant is primarily eliminated in unchanged form (˜77% of the dose) with no major metabolites (i.e., >10%) detected in plasma.

Excretion

The elimination half-life of rimegepant is approximately 11 hours in healthy subjects. Following oral administration of [¹⁴C]-rimegepant to healthy male subjects, 78% of the total radioactivity was recovered in feces and 24% in urine. Unchanged rimegepant is the major single component in excreted feces (42%) and urine (51%).

Specific Populations

Renal Impairment

In a dedicated clinical study comparing the pharmacokinetics of rimegepant in subjects with mild (estimated creatinine clearance [CLcr] 60-89 mL/min), moderate (CLcr 30-59 mL/min), and severe (CLcr 15-29 mL/min) renal impairment to that with normal subjects (healthy matched control), the exposure of rimegepant following single 75 mg dose was approximately 40% higher in subjects with moderate renal impairment. However, there was no clinically meaningful difference in the exposure of rimegepant in subjects with severe renal impairment compared to subjects with normal renal function (CLcr>=90mL/min). NURTEC ODT has not been studied in patients with end-stage renal disease (CLcr<15 mL/min) [see Use in Specific Populations].

Hepatic Impairment

In a dedicated clinical study comparing the pharmacokinetics of rimegepant in subjects with mild, moderate, and severe hepatic impairment to that with normal subjects (healthy matched control), the exposure of rimegepant (Cₘₐₓ and AUC) following a single 75 mg dose was approximately 2-fold higher in subjects with severe impairment (Child-Pugh class C). There were no clinically meaningful differences in the exposure of rimegepant in subjects with mild (Child-Pugh class A) and moderate hepatic impairment (Child-Pugh class B) compared to subjects with normal hepatic function [see Use in Specific Populations].

Other Specific Populations

No clinically significant differences in the pharmacokinetics of rimegepant were observed based on age, sex, race/ethnicity, body weight, or CYP2C9 genotype [see Clinical Pharmacology].

Drug Interaction Studies

In Vitro Studies

Enzymes

Rimegepant is a substrate of CYP3A4 and CYP2C9 [see In Vivo Studies]. Rimegepant is not an inhibitor of CYP1A2, 2B6, 2C9, 2C19, 2D6, or UGT1A1 at clinically relevant concentrations. However, rimegepant is a weak inhibitor of CYP3A4 with time-dependent inhibition. Rimegepant is not an inducer of CYP1A2, CYP2B6, or CYP3A4 at clinically relevant concentrations.

Transporters

Rimegepant is a substrate of P-gp and BCRP. Concomitant administration of inhibitors of P-gp or BCRP may increase the exposure of rimegepant [see Drug Interactions]. No dedicated drug interaction study was conducted to assess their effects on the pharmacokinetics of rimegepant.

Rimegepant is not a substrate of OATP1B1 or OATP1B3. Considering its low renal clearance, rimegepant was not evaluated as a substrate of the OAT1, OAT3, OCT2, MATE1, or MATE2-K.

Rimegepant is not an inhibitor of P-gp, BCRP, OAT1, or MATE2-K at clinically relevant concentrations. It is a weak inhibitor of OATP1B1 and OAT3. Rimegepant is an inhibitor of OATP1B3, OCT2, and MATE1. No clinical drug interactions are expected for NURTEC ODT with these transporters at clinically relevant concentrations.

In Vivo Studies

CYP3A4 Inhibitors

In a dedicated drug interaction study, concomitant administration of 75 mg rimegepant (single dose) with itraconazole, a strong CYP3A4 inhibitor, at steady state resulted in increased exposures of rimegepant (AUC by 4-fold and Cₘₐₓ by ˜1.5-fold) [see Drug Interactions]. No dedicated drug interaction study was conducted to assess the effect of concomitant administration of a weak inhibitor of CYP3A4 on the pharmacokinetics of rimegepant. The concomitant administration of rimegepant with a moderate inhibitor of CYP3A4 may increase rimegepant exposures (AUC) by less than 2-fold [see Drug Interactions]. Concomitant administration of rimegepant with a weak inhibitor of CYP3A4 is not expected to have a clinically significant impact on rimegepant exposures.

CYP3A Inducers

In a dedicated drug interaction study, concomitant administration of 75 mg rimegepant (single dose) with rifampin, a strong CYP3A4 inducer, at steady state resulted in decreased exposures of rimegepant (AUC by 80% and Cₘₐₓ by 64%), which may lead to loss of efficacy [see Drug Interactions]. No dedicated drug interaction study was conducted to assess the effect of concomitant administration of a moderate or weak inducer of CYP3A4 on the pharmacokinetics of rimegepant. Since rimegepant is a moderately sensitive substrate for CYP3A4, drugs that are moderate inducers of CYP3A4 can also cause significant reduction in rimegepant exposure resulting in loss of efficacy [see Drug Interactions]. Clinically significant interaction is not expected with concomitant administration of weak inducers of CYP3A4 and rimegepant.

CYP2C9 Inhibitors

In a dedicated drug interaction study, concomitant administration of 75 mg rimegepant (single dose) with fluconazole, a combined moderate CYP3A4 and CYP2C9 inhibitor, resulted in increased exposures of rimegepant (AUC by 1.8-fold) with no relevant effect on Cₘₐₓ. Rimegepant is primarily metabolized by CYP3A4 and to a lesser extent by CYP2C9. Increase in the exposure of rimegepant can be attributed to combined inhibition of CYP2C9 and CYP3A4 with fluconazole administration suggesting a minor contribution from CYP2C9. Thus, CYP2C9 inhibition alone is not expected to significantly affect rimegepant exposures.

Other Drugs

No significant pharmacokinetic interactions were observed when rimegepant was concomitantly administered with oral contraceptives (norelgestromin, ethinyl estradiol), midazolam (a sensitive CY3A4 substrate), or sumatriptan [see Clinical Pharmacology].

Pharmacogenomics

CYP2C9 activity is reduced in individuals with genetic variants such as the CYP2C9*2 and CYP2C9*3 alleles. Rimegepant Cₘₐₓ and AUC_{0-inf} were similar in CYP2C9 intermediate metabolizers (i.e., *1/*2, *2/*2, *1/*3, n=43) as compared to normal metabolizers (i.e., *1/*1, N=72). Adequate PK data are not available from CYP2C9 poor metabolizers (i.e., *2/*3). Since the contribution of CYP2C9 to rimegepant metabolism is considered minor, CYP2C9 polymorphism is not expected to significantly affect its exposure.

### Nonclinical Toxicology

Carcinogenesis, Mutagenesis, Impairment of Fertility

Carcinogenesis

Oral administration of rimegepant to Tg.rasH2 mice (0, 10, 100, or 300 mg/k/day) for 26 weeks and to rats (0, 5, 20, or 45 mg/kg/day) for 91-100 weeks resulted in no evidence of drug-induced tumors in either species. In rats, the plasma exposure (AUC) at the highest dose tested (45 mg/kg/day) was approximately 30 times that in humans at the maximum recommended human dose (MRHD) of 75 mg/day.

Mutagenesis

Rimegepant was negative in in vitro (bacterial reverse-mutation, chromosomal aberration in Chinese hamster ovary cells) and in vivo (rat micronucleus) assays.

Impairment of Fertility

Oral administration of rimegepant (0, 30, 60, or 150 mg/kg/day) to male and female rats prior to and during mating and continuing in females to gestation day (GD) 7 resulted in uterine atrophy at all doses and reduced fertility at the highest dose tested. In a second fertility study testing lower doses (0, 5, 15, or 25 mg/kg/day), no adverse effects on fertility, uterine histopathology, or early embryonic development were observed. The no-effect dose for impairment of fertility and early embryonic development in rats (25 mg/kg/day) was associated with plasma drug exposures (AUC) approximately 15 times that in humans at the MRHD.

### Clinical Studies

The efficacy of NURTEC ODT for the acute treatment of migraine with and without aura in adults was demonstrated in a randomized, double-blind, placebo-controlled trial: Study 1 (NCT03461757). The study randomized patients to 75 mg of NURTEC ODT (N=732) or placebo (N=734). Patients were instructed to treat a migraine of moderate to severe headache pain intensity. Rescue medication (i.e., NSAIDs, acetaminophen, and/or an antiemetic) was allowed 2 hours after the initial treatment. Other forms of rescue medication such as triptans were not allowed within 48 hours of initial treatment. Approximately 14% of patients were taking preventive medications for migraine at baseline. None of the patients in Study 1 were on concomitant preventive medication that act on the CGRP pathway.

The primary efficacy analyses were conducted in patients who treated a migraine with moderate to severe pain. NURTEC ODT 75 mg demonstrated an effect on pain freedom and most bothersome symptom (MBS) freedom at two hours after dosing, compared to placebo. Pain freedom was defined as a reduction of moderate or severe headache pain to no headache pain, and MBS freedom was defined as the absence of the self-identified MBS (i.e., photophobia, phonophobia, or nausea). Among patients who selected an MBS, the most commonly selected symptom was photophobia (54%), followed by nausea (28%), and phonophobia (15%).

In Study 1, the percentage of patients achieving headache pain freedom and MBS freedom two hours after a single dose was statistically significantly greater in patients who received NURTEC ODT compared to those who received placebo (Table 1).

TABLE 1
Migraine Efficacy Endpoints for Study 1

| | Study 1 | |
|---|---|---|
| | NURTEC ODT | |
| | 75 mg | Placebo |
| Pain Free at 2 hours | | |
| n/N* | 142/669 | 74/682 |
| % Responders | 21.2 | 10.9 |
| Difference from placebo (%) | 10.3 | |
| p-value | | <0.001 |
| MBS Free at 2 hours | | |
| n/N* | 235/669 | 183/682 |
| % Responders | 35.1 | 26.8 |
| Difference from placebo (%) | 8.3 | |
| p-value | | 0.001 |

*n = number of responders/N = number of patients in that treatment group

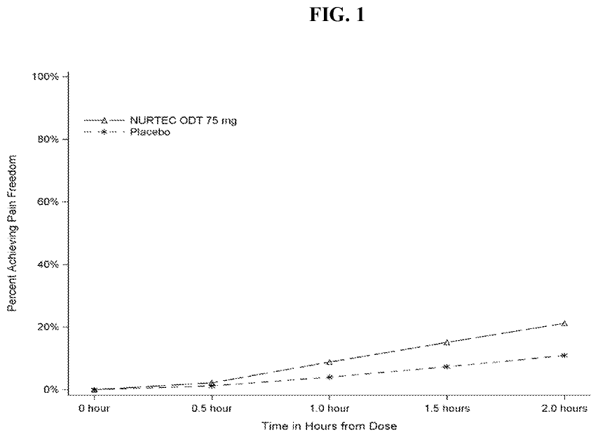

FIG. 1 presents the percentage of patients achieving migraine pain freedom within 2 hours following treatment in Study 1.

In Study 1, statistically significant effects of NURTEC ODT compared to placebo were demonstrated for the additional efficacy endpoints of pain relief at 2 hours, sustained pain freedom 2-48 hours, use of rescue medication within 24 hours, and the percentage of patients reporting normal function at two hours after dosing (Table 2). Pain relief was defined as a reduction in migraine pain from moderate or severe severity to mild or none. The measurement of the percentage of patients reporting normal function at two hours after dosing was derived from a single item questionnaire, asking patients to select one response on a 4-point scale; normal function, mild impairment, severe impairment, or required bedrest.

TABLE 2
Additional Migraine Efficacy Endpoints in Study 1

| | Study 1 | |
|---|---|---|
| | NURTEC ODT | |
| | 75 mg | Placebo |
| Pain Relief at 2 hours | | |
| n/N* | 397/669 | 295/682 |
| % Responders | 59.3 | 43.3 |
| Difference from placebo | 16.1 | |
| p-value | | <0.001 |
| Sustained Pain Freedom 2-48 hours | | |
| n/N* | 90/669 | 37/682 |
| % Responders | 13.5 | 5.4 |
| Difference from placebo | 18.0 | |
| p-value | | <0.001 |
| Use of Rescue Medication within | | |
| 24 hours** | | |
| n/N* | 95/669 | 199/682 |
| % Responders | 14.2 | 29.2 |
| Difference from placebo | −15.0 | |
| p-value | | <0.001 |
| Percentage of Patients Reporting | | |
| Normal Function at 2 hours | | |
| n/N* | 255/669 | 176/682 |
| % Responders | 38.1 | 25.8 |
| Difference from placebo | 12.3 | |
| p-value | | <0.001 |

*n = number of responders/N = number of patients in that treatment group
**This analysis includes only the use of NSAIDs, acetaminophen, or antiemetics, within 24 hours post-dose; the use of triptans, or other acute migraine medication, was not allowed.

The incidence of photophobia and phonophobia was reduced following administration of

NURTEC ODT 75 mg as compared to placebo.

### How Supplied/Storage and Handling

How Supplied

NURTEC ODT 75 mg may be in a form of orally disintegrating tablets, which may be white to off-white, which may be circular, which may be debossed with -the symbol and which may be supplied in cartons containing a blister pack of 8 orally disintegrating tablets. Each ODT may contain 75 mg rimegepant.

NDC: 72618-3000-2

Storage and Handling

NURTEC ODT is stored at controlled room temperature, 20° C. to 25° C. (68° F. to 77° F.); with excursions permitted between 15° C. to 30° C. (59° F. to 86° F.) [see USP controlled room temperature].

### Patient Counseling Information

Patients should be advised to read the FDA-approved patient labeling (Patient Information).

Handling of Orally Disintegrating Tablets Packaging

Patients should be instructed not to remove the blister from the outer aluminum pouch until ready to use the orally disintegrating tablet inside [see Dosage and Administration].

Hypersensitivity Reactions

Patients should be informed about the signs and symptoms of hypersensitivity reactions and that these reactions can occur days after administration of NURTEC ODT. Patients should be advised to contact their healthcare provider immediately if signs or symptoms of hypersensitivity reactions occur [see Warnings and Precautions].

### Highlights of Prescribing Information

Indications and Usage. NURTEC ODT is a calcitonin gene-related peptide receptor antagonist indicated for the acute treatment of migraine with or without aura in adults. NURTEC ODT is not indicated for the preventive treatment of migraine.

Dosage and Administration. The recommended dose is 75 mg taken orally, as needed. The maximum dose in a 24-hour period is 75 mg. The safety of treating more than 15 migraines in a 30-day period has not been established.

Dosage Forms and Strength. NURTEC ODT orally disintegrating tablets: 75 mg.

Contraindications. Patients with a history of hypersensitivity reaction to rimegepant, NURTEC ODT, or to any of its components.

Warnings and Precautions. Hypersensitivity Reactions: If a serious hypersensitivity reaction occurs, discontinue NURTEC ODT and initiate appropriate therapy. Severe hypersensitivity reactions have included dyspnea and rash, and can occur days after administration.

Adverse Reactions. The adverse reaction reported in >1% of patients treated with NURTEC ODT is nausea.

Drug Interactions.
Strong CYP3A4 Inhibitors: Avoid concomitant administration.
Moderate CYP3A4 Inhibitors: Avoid another dose within 48 hours when administered with a moderate CYP3A4 inhibitor.
Strong and Moderate CYP3A Inducers: Avoid concomitant administration.
Inhibitors of P-gp or BCRP: Avoid concomitant administration.

Use in Specific Populations. Exposures were significantly higher in subjects with severe hepatic impairment. Avoid use in patients with severe hepatic impairment (Child-Pugh C).

Throughout this application, various publications are referenced by author name and date, or by patent number or patent publication number. The disclosures of these publications are hereby incorporated in their entireties by reference into this application in order to more fully describe the state of the art as known to those skilled therein as of the date of the invention described and claimed herein. However, the citation of a reference herein should not be construed as an acknowledgement that such reference is prior art to the present invention.

Those skilled in the art will recognize, or be able to ascertain using no more than routine experimentation, numerous equivalents to the specific procedures described herein. Such equivalents are considered to be within the scope of this invention and are covered by the following claims. For example, pharmaceutically acceptable salts other than those specifically disclosed in the description and Examples herein can be employed. Furthermore, it is intended that specific items within lists of items, or subset groups of items within larger groups of items, can be combined with other specific items, subset groups of items or larger groups of items whether or not there is a specific disclosure herein identifying such a combination.

1. A method of treating migraine in a patient in need thereof, comprising orally administering to the patient a fast-dispersing pharmaceutical composition comprising a pharmaceutically acceptable carrier and a therapeutically effective amount of rimegepant, or a pharmaceutically acceptable salt thereof.

2. The method according to claim 1, wherein administering the pharmaceutical composition results in migraine pain freedom within 2 hours of the time of administration.

3. The method according to claim 1, wherein administering the pharmaceutical composition results in about 21.2% of patients achieving migraine pain freedom within 2 hours of the time of administration.

4. The method according to claim 1, wherein administering the pharmaceutical composition results in freedom from most bothersome symptoms within 2 hours of the time of administration.

5. The method according to claim 1, wherein administering the pharmaceutical composition results in about 35.1% of patients achieving freedom from most bothersome symptoms within 2 hours of the time of administration.

6. The method according to claim 5, wherein the most bothersome symptoms comprise photophobia, nausea, phonophobia, or a combination thereof.

7. The method according to claim 1, wherein administering the pharmaceutical composition results in pain relief within 90 minutes of the time of administration.

8. The method according to claim 1, wherein administering the pharmaceutical composition results in about 49.6% of patients achieving pain relief within 90 minutes of the time of administration.

9. The method according to claim 1, wherein administering the pharmaceutical composition results in achieving pain relief within 2 hours of the time of administration.

10. The method according to claim 1, wherein administering the pharmaceutical composition results in about 59.3% of patients achieving pain relief within 2 hours of the time of administration.

11. The method according to claim 1, wherein administering the pharmaceutical composition results in sustained pain relief within 2 to 48 hours of the time of administration.

12. The method according to claim 1, wherein administering the pharmaceutical composition results in about 42.2% of patients achieving sustained pain relief within 2 to 48 hours of the time of administration.

13. The method according to claim 1, wherein administering the pharmaceutical composition results in sustained pain freedom within 2 to 48 hours of the time of administration.

14. The method according to claim 1, wherein administering the pharmaceutical composition results in about 13.5% of patients achieving sustained pain freedom within 2 to 48 hours of the time of administration.

15. The method according to claim 1, wherein administering the pharmaceutical composition results in reduced usage of rescue medication within 24 hours of the time of administration.

16. The method according to claim 1, wherein administering the pharmaceutical composition results in about 14.2% of patients using rescue medication within 24 hours of the time of administration.

17. The method according to claim 1, wherein administering the pharmaceutical composition results in normal function within 2 hours of the time of administration.

18. The method according to claim 1, wherein administering the pharmaceutical composition results in about 38.1% of patients reporting normal function within 2 hours of the time of administration.

19. The method according to claim 1, wherein the daily dose of rimegepant is about 75 mg.

20. The method according to claim 1, wherein the pharmaceutical composition is administered to a patient up to 15 times in a 30-day period.

21. The method according to claim 1, wherein the pharmaceutically acceptable salt or rimegepant is rimegepant hemi sulfate.

22. The method according to claim 1, wherein the daily dose of rimegepant hemisulfate is about 85.65 mg.

23. The method according to claim 1, wherein the pharmaceutical composition further includes benzyl alcohol, eucalyptol, gelatin, limonene, mannitol, menthol, menthone, menthyl acetate, sucralose, vanillin, or a combination thereof.

24. The method according to claim 1, wherein about 1.3% or less of patients who received the pharmaceutical composition experience nausea.

25. The method according to claim 1, wherein about 0.15% or less of patients who received the pharmaceutical composition experience hypersensitivity.

26. The method according to claim 25, wherein the hypersensitivity comprises dyspnea, rash, or a combination thereof.
